(19) United States
(12) Patent Application Publication
(10) Pub. No.: US 2026/0048217 A1
(43) Pub. Date: Feb. 19, 2026

(54) CONTROL UNIT FOR A VENTILATOR

(71) Applicant: Loewenstein Medical Technology S.A., Luxembourg (LU)

(72) Inventors: Peter KREMEIER, Karlsruhe (DE); Gerardo Tusman, Mar del Plata (AR)

(21) Appl. No.: 19/278,022

(22) Filed: Jul. 23, 2025

(30) Foreign Application Priority Data
Aug. 14, 2024 (DE) 10 2024 123 174.2

(51) Int. Cl. A61M 16/00; G16H 40/63

(52) U.S. Cl. CPC A61M 16/024; A61M 16/0003; G16H 40/63

(57) ABSTRACT

A ventilator comprises a respiratory air connector connected to the respiratory system of a patient; an actuation system for providing a respiratory air flow; a sensor system for creating measurement data relating to the ventilation. A control unit for the ventilator is configured to carry out: creating a control signal for controlling the actuation system such that a ventilation maneuver is performed, within which at least one section of the respiratory system is opened/closed in an inhalation/exhalation phase; receiving the measurement data indicating a pressure-dependent curve of an amount of a respiratory gas inhaled/exhaled by the patient as a function of a pressure of the respiratory air; determining an opening/closing pressure corresponding to a pressure of the respiratory air at which at least one section of the respiratory system is opened/closed, by evaluating a section of the pressure-dependent curve that relates to the inhalation/exhalation phase.

### CROSS-REFERENCE TO RELATED APPLICATIONS

The present application claims priority under 35 U.S.C. § 119 of German Patent Application No. 10 2024 123 174.2, filed Aug. 14, 2024, the entire disclosure of which is expressly incorporated by reference herein.

### BACKGROUND OF THE INVENTION

### 1. Field of the Invention

The invention relates to a control unit for a ventilator. The invention also relates to a computer program that is executable by the control unit, a corresponding computer-readable medium and a ventilator having such a control unit.

### 2. Discussion of Background Information

Under certain circumstances, artificially ventilated patients may experience a recurrent collapse of the lung or part of the lung during exhalation. In a worst-case scenario, this may cause local injuries to the lung, which may adversely affect the prognosis for the patient. Thus, ventilation should be implemented in such a way that such lung collapses are avoided where possible.

Moreover, in the context of maneuvers for lung recruitment, it could be shown that it is important to identify not only a collapse of the lung but also a closure of the airways.

Such closure of the airways may lead to an interruption of the respiratory air flow between the proximal airway opening and the distal (smaller) airway structures and/or the distal alveolar structures. As a consequence, the lung can only be re-inflated once the airways have been opened again. A pressure required to this end may also be referred to as airways opening pressure. However, the pressure in the airways may differ significantly from the pressure in the alveoli—even in the event of end-expiratory closure. This may lead to an incorrect assessment of the ventilation situation. Hence there is a need for a reliable and easy-to-implement method for identifying such pressures.

In view of the foregoing, it would be advantageous to have available a control unit that allows reliable identification of the opening and/or closing of at least one section of the respiratory system of a patient when they are ventilated. It further would be advantageous to have available a corresponding computer program, a corresponding computer-readable medium and a corresponding ventilator.

### SUMMARY OF THE INVENTION

In a first aspect the invention provides a control unit for a ventilator. The ventilator comprises: a respiratory air connector, to which the respiratory system of a patient is connected such that a ventilation of the patient with respiratory air is rendered possible; an actuation system for providing a respiratory air flow at the respiratory air connector; a sensor system for creating measurement data in relation to the ventilation. The control unit is configured to carry out the following method: creating a control signal for controlling the actuation system such that a ventilation maneuver is performed, within the scope of which at least one section of the respiratory system is opened in an inhalation phase and/or is closed (or collapses) in an exhalation phase; receiving the measurement data, wherein the measurement data indicate a pressure-dependent curve of an amount of at least one respiratory gas inhaled by the patient in the inhalation phase and/or exhaled in the exhalation phase, as a function of a pressure of the respiratory air; determining an opening pressure, which corresponds to a pressure of the respiratory air at which at least one section of the respiratory system is opened, by evaluating a section of the pressure-dependent curve that relates to the inhalation phase, and/or determining a closing pressure, which corresponds to a pressure of the respiratory air at which at least one section of the respiratory system is closed (or collapses), by evaluating a section of the pressure-dependent curve that relates to the exhalation phase.

Surprisingly, trials have shown that the curve of the carbon dioxide or oxygen partial pressure over the ventilation pressure in a corresponding (pressure-based) capnogram or oxigram has characteristic knees that significantly correlate with the opening and closing pressures as may be determined in a conventional bedside lung recruitment maneuver with subsequent PEEP titration. In other words, the opening or closing pressures may be read directly from such a capnogram or oxigram without necessarily having to consult additional information in order to check the plausibility. This represents a significant advantage in relation to accuracy, reliability and efficiency vis-à-vis conventional recruitment or titration methods.

In contrast to complex probability-based calculation methods for assessing the airways opening pressure and the lung mechanics during ventilation, such a method allows an accurate and reliable determination of the opening or closing pressure with significantly reduced computational outlay. A further advantage lies in the fact that a time-consuming PEEP titration over a plurality of breaths may be omitted. Moreover, this can reduce the risk of a lung overexpansion, as may occur due to a ventilation pressure that is elevated over a relatively long period of time.

The control unit may comprise elements for data processing. The elements for data processing may be implemented as hardware and/or software and/or may comprise a processor. The processor may be configured to carry out the (computer-implemented) method. In addition to the processor, the control unit may comprise at least one of the following data-processing elements: a memory, a bus system for communicating data between the memory and the processor, and a data communications interface for communicating data with peripherals wirelessly and/or in wired fashion. Alternatively, the control unit may be implemented exclusively as hardware, for example in the form of an ASIC component or FPGA component.

“Respiratory air” may be understood to mean a single respiratory gas or a mixture of a plurality of respiratory gases for ventilating the patient. “Respiratory gas” may be understood to mean e.g. carbon dioxide, oxygen, nitrogen, water vapor or an inhalational anesthetic.

In general, a “ventilation maneuver” may be understood to mean a maneuver in which, as a consequence of an appropriately controlled pressure and/or volumetric flow rate of the respiratory air, at least one section of the respiratory system, for example at least one section of the airways and/or of the lung, more precisely of the alveoli, opens during inhalation (also referred to as recruitment) and/or closes (or collapses) during exhalation. The ventilation maneuver may be performed during a single breath or during a plurality of breaths, for example a plurality of directly successive breaths.

Closure of the respiratory system at least in sections should be understood to mean that the closure does not occur actively but (purely) passively on account of the surface tension and the restoring forces of the lung tissue and the thorax. It is possible to counteract this avalanche-like system in a targeted manner during ventilation, for example in such a way that a corresponding increase or reduction in the time duration and/or in the pressure range of the closure is brought about. Hence, the term “closure” should be understood to mean a collapse in particular.

It is possible that the control signal is created under additional use of the measurement data (or at least some of the measurement data). This allows the ventilation maneuver to be controlled on the basis of the current state of the patient.

An “amount” may be understood to mean a partial pressure or a concentration of the respective respiratory gas in the respiratory air flow and/or in the blood of the patient.

The measurement data may have been created using the sensor system when the ventilation maneuver was performed. For example, the measurement data used to determine the opening pressure, i.e., the measurement data indicating the inhalation-phase-related section of the pressure-dependent curve, may have been created in the inhalation phase of the ventilation maneuver. Accordingly, the measurement data used to determine the closing pressure, i.e. the measurement data indicating the exhalation-phase-related section of the pressure-dependent curve, may have been created in the exhalation phase of the ventilation maneuver. In addition, at least one other section of the pressure-dependent curve may be evaluated for the purpose of determining the opening pressure and/or the closing pressure.

It is possible that the measurement data are created during a single breath and/or are re-created and/or re-received for each breath. Expressed differently, the pressure-dependent curve may relate to a single breath and/or may be updated for each breath. Depending on the respective respiratory gas, the pressure-dependent curve may for example also be referred to as pressure-based or barometric capnogram or oxigram.

In a second aspect the invention provides a ventilator. The ventilator comprises: a respiratory air connector for connecting the respiratory system of a patient such that the ventilation of the patient with respiratory air is rendered possible; an actuation system for providing a respiratory air flow at the respiratory air connector; a sensor system for creating measurement data in relation to the ventilation; and a control unit as described above and below.

A “ventilator” may for example be understood to mean a device for invasive and/or non-invasive ventilation of the patient and/or an anesthesia device.

The respiratory air connector may be connectable to the respiratory system by way of one or more ventilation tubes and/or by way of a suitable patient interface, for example a mask, a nasal cannula or a tube.

For example, the actuation system may comprise one or more blowers and/or one or more electrically controllable valves.

For example, the sensor system may be configured to acquire at least one of the following quantities: the amount of the respective respiratory gas inhaled by the patient in the inhalation phase and/or exhaled in the exhalation phase, the pressure of the respiratory air, a volume of the respiratory air, a volumetric flow rate of the respiratory air. The sensor system may comprise one or more sensors. The sensor or at least some of the sensors may be arranged in a main stream and/or in a secondary stream of the respiratory air inhaled and/or exhaled by the patient.

In a third aspect the invention provides a computer program for operating a ventilator as described above and below. The computer program comprises commands that prompt the control unit, for example a processor of the control unit, to carry out the following method when the computer program is executed by the control unit: creating a control signal for controlling the actuation system such that a ventilation maneuver is performed, within the scope of which at least one section of the respiratory system is opened in an inhalation phase and/or is closed (or collapses) in an exhalation phase; receiving the measurement data, wherein the measurement data indicate a pressure-dependent curve of an amount of at least one respiratory gas inhaled by the patient in the inhalation phase and/or exhaled in the exhalation phase, as a function of a pressure of the respiratory air; determining an opening pressure, which corresponds to a pressure of the respiratory air at which at least one section of the respiratory system is opened, by evaluating a section of the pressure-dependent curve that relates to the inhalation phase, and/or determining a closing pressure, which corresponds to a pressure of the respiratory air at which at least one section of the respiratory system is closed (or collapses), by evaluating a section of the pressure-dependent curve that relates to the exhalation phase.

In a fourth aspect the invention provides a computer-readable medium on which a computer program, as described above and below, is stored.

The computer-readable medium may be a transitory or non-transitory data medium. For example, the computer-readable medium may be a hard disk, a USB (universal serial bus) storage device, a RAM (random-access memory), a ROM (read-only memory), an EPROM (erasable programmable read-only memory), an EEPROM (electrically erasable programmable read-only memory), a flash memory or a combination of at least two of these examples. The computer-readable medium may also be a data communications network that allows program code to be downloaded (e.g. via the Internet), or a cloud.

It should be noted that features of the control unit described above and below can also be features of the computer program and/or the computer-readable medium (and vice versa).

Various embodiments of the invention are described below. These embodiments should not be understood as restricting the scope of the invention.

According to an embodiment, the opening pressure may comprise an airways opening pressure that corresponds to a pressure of the respiratory air at which the (previously at least partially closed) airways of the patient are at least partially opened. In addition to that or in an alternative, the opening pressure may comprise a lung opening pressure that corresponds to a pressure of the respiratory air at which the lung of the patient (that was previously at least partially collapsed) is at least partially opened or recruited.

According to an embodiment, the closing pressure may comprise a lung closing pressure that corresponds to a pressure of the respiratory air at which the lung of the patient (that was previously at least partially opened or recruited) is at least partially closed, i.e., collapses at least in part. In addition to that or in an alternative, the closing pressure may comprise an airways closing pressure that corresponds to a pressure of the respiratory air at which the airways of the patient (that were previously at least partially opened) are at least partially closed (or collapse at least in part).

According to an embodiment, the control signal may be created such that an inhalation pressure which corresponds to a pressure of the respiratory air in the inhalation phase is increased in a plurality of successive time steps. In this case, the inhalation pressure in each time step may be higher than in the respective preceding time step. Expressed differently, it is possible that the inhalation pressure is continuously increased to a predetermined end value starting from a predetermined initial value. In addition to that or in an alternative, the inhalation pressure may follow a predetermined inhalation pressure curve. In this case, the inhalation pressure curve may increase linearly at least in part. It is expedient for the initial value to correspond to an inhalation pressure at which at least one section of the respiratory system is closed. For example, the initial value of the inhalation pressure may be zero or correspond to the respective ambient pressure. By contrast, the end value should correspond to an inhalation pressure at which at least the majority of the lung is opened or recruited without being excessively expanded. For example, the end value of the inhalation pressure may be no more than about 30 cm H₂O, no more than about 40 cm H₂O, no more than about 50 cm H₂O or no more than about 60 cm H₂O. However, other initial and/or end values of the inhalation pressure are also possible, depending on the state of the respective patient.

According to an embodiment, the control signal may be created such that a volumetric flow rate of the respiratory air in the exhalation phase follows a predetermined (e.g. time-dependent) volumetric flow rate curve. Expressed differently, the ventilation in the exhalation phase may be flow controlled. The volumetric flow rate curve may be at least partially constant and/or at least partially increase and/or at least partially drop off. This allows a more accurate evaluation of the pressure-dependent curve in comparison with a pressure-controlled exhalation phase.

According to an embodiment, a volumetric flow rate of the respiratory air in the exhalation phase may be from about 0.02 l/s to about 0.20 l/s, preferably from about 0.05 l/s to about 0.15 l/s and particularly preferably about 0.10 l/s. Alternatively, the volumetric flow rate may be at least about 0.02 l/s, preferably at least about 0.05 l/s, and/or no more than about 0.20 l/s, preferably no more than about 0.15 l/s. This allows a particularly accurate evaluation of the pressure-dependent curve on account of the time window being significantly increased in comparison with a pressure-controlled exhalation phase.

According to an embodiment, the control signal may be created such that an exhalation pressure which corresponds to a pressure of the respiratory air in the exhalation phase is decreased in a plurality of successive time steps. In this case, the exhalation pressure in each time step may be lower than in the respective preceding time step. Expressed differently, it is possible that the exhalation pressure is continuously decreased to a predetermined end value starting from a predetermined initial value. In addition to that or in an alternative, the exhalation pressure may follow a predetermined exhalation pressure curve. In this case, the exhalation pressure curve may drop off linearly at least in part. The end value of the exhalation pressure may be e.g. zero or correspond to the respective ambient pressure. It is possible that the initial value of the exhalation pressure corresponds to an end value of an inhalation pressure at the end of the preceding inhalation phase and/or that the end value of the exhalation pressure corresponds to an initial value of an inhalation pressure at the start of a subsequent inhalation phase. The end value of the exhalation pressure may for example also be referred to as positive end-expiratory pressure, abbreviated to PEEP. Accordingly, the initial value of the inhalation pressure may correspond to the PEEP of a preceding breath.

According to an embodiment, determination of the opening pressure and/or of the closing pressure may comprise: identifying a characteristic knee in the pressure-dependent curve; determining a pressure of the respiratory air associated with the characteristic knee, in order to determine the opening pressure and/or the closing pressure. Expressed differently, the determination of the opening pressure and/or closing pressure may comprise an identification of a characteristic change in the gradient of the pressure-dependent curve.

If the airways are closed and the inhalation pressure reaches a critical threshold, then the airways open suddenly such that fresh, i.e. oxygen-rich, respiratory air may flow into the lung. This leads to a dilution of the consumed, i.e. carbon-dioxide-rich, respiratory air that remained in the respiratory system during the preceding exhalation, and this is expressed in a correspondingly significant drop in the carbon dioxide partial pressure in the respiratory air flow. The influence of this sudden opening can be identified as a characteristic knee in an appropriate pressure-based capnogram for example. Hence, the pressure of the respiratory air associated with this knee may be considered to be the airways opening pressure.

According to an embodiment, the characteristic knee may be identified on the basis of a characteristic absolute-value reduction in for example an average gradient of the pressure-dependent curve with increasing ventilation duration. This allows an accurate and reliable identification of the opening or closing pressure even in the case of a strongly fluctuating state of the patient.

For example, the characteristic knee associated with the opening pressure, in particular the airways opening pressure, may correspond to an absolute-value reduction in the (average) gradient of the pressure-dependent curve by at least about 50 percent or by at least about 70 percent in the first third of the inhalation phase.

For example, a section of the pressure-dependent curve over an inhalation pressure range of from about 0 to about 20 cm H₂O or from about 5 to about 15 cm H₂O or from about 10 to about 15 cm H₂O may be evaluated for the purpose of determining the opening pressure.

However, other inhalation pressure ranges are also possible, depending on the state of the respective patient.

For example, a section of the pressure-dependent curve over an exhalation pressure range of from about 0 to about 20 cm H₂O or from about 0 to about 15 cm H₂O or from about 0 to about 10 cm H₂O may be evaluated for the purpose of determining the closing pressure. However, other exhalation pressure ranges are also possible, depending on the state of the respective patient.

According to an embodiment, the inhalation phase and the exhalation phase may be successive phases of a single breath. Expressed differently, the pressure-dependent curve may relate to a single breath.

According to an embodiment, a section of the pressure-dependent curve may be evaluated in the first half or in the first third of the inhalation phase (for example of a single breath) in order to determine the opening pressure. Expressed differently, the opening pressure may be determined by evaluating the pressure-dependent curve tendentially at the start of the inhalation phase rather than at its end. In addition to that or in an alternative, a section of the pressure-dependent curve may be evaluated in the last third of the exhalation phase (for example in the same breath as the inhalation phase) in order to determine the closing pressure. Expressed differently, the closing pressure may be determined by evaluating the pressure-dependent curve tendentially at the end of the exhalation phase rather than at its start.

The division of the exhalation phase into a plurality of phase sections may for example be implemented by evaluating a corresponding volumetric capnogram or oxigram. In this case, the respective volume-dependent curve may for example be subdivided into at least three successive phase sections that are characteristic for a single breath. The phase sections may differ significantly in terms of their length and/or in terms of the (e.g., average) gradient of the volume-dependent curve.

For example, in the case of a volumetric capnogram, a first phase section may extend from the start of exhalation to a first point at which the rate of change of the second derivative of the volume-dependent curve reaches its maximum or the third derivative of the volume-dependent curve reaches its left-hand maximum. A second phase section may extend from the first point to a second point at which the third derivative of the volume-dependent curve reaches its right-hand maximum. A third phase section may extend from the second point to the end of exhalation. In this case, the term “volume-dependent curve” may also be understood to mean a suitable approximation. The first phase section may be the earliest phase of exhalation (for example about 10% to about 12% of the entire breath), in which hardly any or no carbon dioxide is contained in the respiratory air. The second phase section may be a phase of greatest (average) increase in the amount of carbon dioxide in the respiratory air flow (for example about 15% to about 18% of the entire breath). The third phase section may be a phase in which the amount of carbon dioxide—in contrast to the preceding phase sections—is predominantly determined by the gases coming from the alveoli (for example about 70% to about 75% of the entire breath).

For example, the phase sections may be determined approximately from the (e.g. measured) volume-dependent curve using Fowler's method and/or the Levenberg-Marquardt algorithm. A “Levenberg-Marquardt algorithm” may be understood to mean a specific numerical optimization algorithm for performing nonlinear curve fitting with the aid of the least squares method. The algorithm may be considered to be a combination of the Gauss-Newton method with a regularization technique that forces decreasing functional values. This allows for a more accurate and computationally efficient approximation than an implementation of the conventional Fowler's method, to be precise even in the case of relatively strong fluctuations in the volume-dependent curve between successive breaths and/or between different patients.

Alternatively, the phase sections may be determined accordingly on the basis of the volumetric oxigram.

According to an embodiment, the measurement data may further indicate a volume-dependent curve of the amount of at least one respiratory gas inhaled by the patient in the inhalation phase and/or exhaled in the exhalation phase, as a function of a volume of the respiratory air. In this case, the opening pressure may furthermore be determined by evaluating a section of the volume-dependent curve that relates to the inhalation phase. In addition to that or in an alternative, the closing pressure may furthermore be determined by evaluating a section of the volume-dependent curve that relates to the exhalation phase. For example, each point of the volume-dependent curve may be associated with a specific fraction of an overall volume of the respiratory air (comprising the respective respiratory gas) inhaled and/or exhaled by the patient during a single breath. Accordingly, the start of the volume-dependent curve may be associated with a volume value of zero, and the end of the volume-dependent curve may be associated with a volume value that equals the overall volume. Such an overall volume may also be referred to as the volume of a breath or a tidal volume. Depending on the respective respiratory gas, the volume-dependent curve may for example also be referred to as volume-based or volumetric capnogram or oxigram. This embodiment allows for an additional check of the plausibility of the results obtained by evaluating the pressure-dependent curve.

According to an embodiment, the measurement data may further indicate a time-dependent curve of a volumetric flow rate of the respiratory air inhaled by the patient in the inhalation phase and/or exhaled in the exhalation phase, as a function of a ventilation duration. In this case, the opening pressure may furthermore be determined by evaluating a section of the time-dependent curve that relates to the inhalation phase. In addition to that or in an alternative, the closing pressure may furthermore be determined by evaluating a section of the time-dependent curve that relates to the exhalation phase. In this case, determining the opening pressure and/or the closing pressure—in a manner similar to the evaluation of the pressure-dependent curve—may comprise: identifying a further characteristic knee in the time-dependent curve; determining a pressure of the respiratory air associated with the further characteristic knee, in order to determine the opening pressure and/or the closing pressure. This embodiment allows for an additional check of the plausibility of the results obtained by evaluating the pressure-dependent curve.

In the case of a (complete) closure of the airways at the start of the inhalation phase, the pressure of the respiratory air increases while the volumetric flow rate of the respiratory air tends to zero. Only once the pressure of the respiratory air is so high that the airways open does the volumetric flow rate start to increase as well. The pressure of the respiratory air associated with this first knee in the time-dependent curve of the volumetric flow rate in the inhalation phase may thus be considered to be the airways opening pressure.

The lung is increasingly filled with respiratory air after the airways were opened. Some distal sections of the lung may still be collapsed at this time. Only once the pressure of the respiratory air is large enough do these still collapsed sections, such as the relatively small airway and alveolar structures, also open up. The volumetric flow rate of the respiratory air correspondingly increases significantly in the process. The pressure of the respiratory air at which the volumetric flow rate of the respiratory air reaches its (local) maximum in the inhalation phase may therefore be considered to be the lung opening pressure. If the lung opening pressure is exceeded during the further course of the inhalation phase, then the volumetric flow rate drops off significantly again because the lung tissue has reached its elastic limit in that case. There may be an unwanted overexpansion of the lung in the case of a further increase in pressure beyond this point.

According to an embodiment, the method may further comprise: determining at least one target value for the pressure of the respiratory air taking into account the opening pressure and/or the closing pressure; using the at least one target value for controlling the actuation system. The target value or target values may for example predetermine a positive end-expiratory pressure, PEEP for short. For example, the target value or target values may be located between the opening pressure and the closing pressure. Hence it is possible to omit a time-consuming titration of the individual PEEP for the respective patient, as is usually performed.

According to an embodiment, the measurement data may comprise (e.g. measured and/or estimated) values for at least one of the following quantities and/or may be based on (e.g. measured and/or estimated) values for at least one of the following quantities: the amount of the at least one respiratory gas inhaled by the patient in the inhalation phase and/or exhaled in the exhalation phase, the pressure of the respiratory air, a volume of the respiratory air, a volumetric flow rate of the respiratory air. For example, the volume of the respiratory air may also be determined by integrating the volumetric flow rate of the respiratory air.

### BRIEF DESCRIPTION OF THE DRAWINGS

Embodiments of the invention are described hereinafter with reference to the accompanying drawings. Neither the description nor the drawings should be understood as restricting the scope of the invention. In the drawings:

FIG. 1 shows a ventilator according to an embodiment of the invention.

FIG. 2 shows a flowchart for explaining a method that may be carried out by a control unit according to an embodiment of the invention.

FIG. 3 shows a time-dependent pressure curve and a time-dependent volumetric flow rate curve when performing a ventilation maneuver using a control unit according to an embodiment of the invention.

FIG. 4 shows a pressure-based capnogram for evaluation by a control unit according to an embodiment of the invention.

FIG. 5 shows a volume-based capnogram for evaluation by a control unit according to an embodiment of the invention.

The drawings are purely schematic and not true to scale. If identical reference signs are used in different drawings, then these reference signs designate identical or identically acting features.

### DETAILED DESCRIPTION OF EXEMPLARY EMBODIMENTS

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show details of the present invention in more detail than is necessary for the fundamental understanding of the present invention, the description in combination with the drawings making apparent to those of skill in the art how the several forms of the present invention may be embodied in practice.

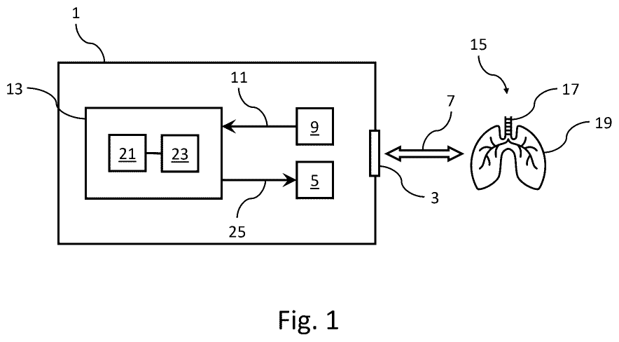

FIG. 1 shows a ventilator 1 which comprises a respiratory air connector 3, an actuation system 5 for providing a respiratory air flow 7 at the respiratory air connector 3 and a sensor system 9 for creating measurement data 11 in relation to at least one measured quantity relevant to ventilation. Moreover, the ventilator 1 comprises a control unit 13 that is connected to the actuation system 5 and the sensor system 9 and serves to control the actuation system 5, for example by making additional use of the measurement data 11.

The actuation system 5 may comprise one or more blowers and/or one or more electrically controllable valves.

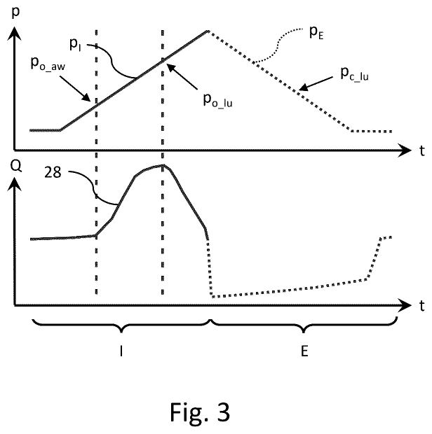

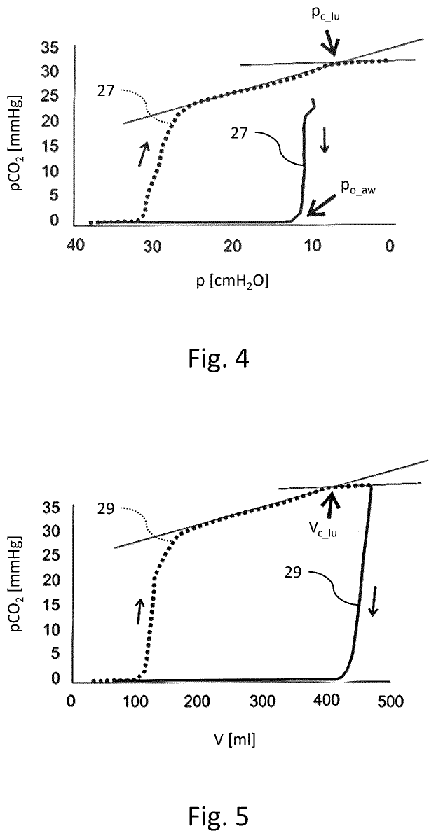

The sensor system 9 may comprise one or more sensors, for example at least one of the following sensors: a carbon dioxide sensor for measuring a carbon dioxide partial pressure pCO₂ (see FIG. 4 and FIG. 5), an oxygen sensor for measuring an oxygen partial pressure, a flow sensor for measuring a volumetric flow rate Q of the respiratory air, a pressure sensor for measuring a pressure p of the respiratory air (see FIG. 3).

The respiratory air connector 3 is connected—for example via one or more ventilation tubes and a suitable patient interface such as e.g. a mask, a nasal cannula or a tube—to the respiratory system 15 of the patient, in particular to the patient's airways 17 and/or the patient's lung 19 such that the patient can be ventilated with respiratory air. The ventilation may be implemented invasively or noninvasively, depending on the application.

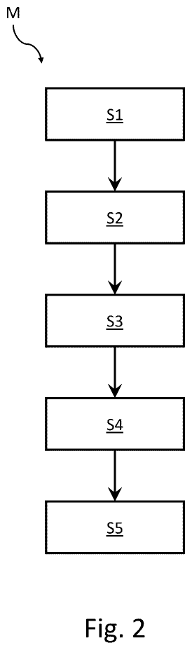

The control unit 13 may comprise a processor 21 and a memory 23, in which a computer program for operating the ventilator 1 may be stored. By executing the computer program, the processor 21 may be configured to carry out the method M for operating the ventilator 1 as set forth below. The workflow of the method M is illustrated in FIG. 2.

In a step S1, a control signal 25 is created for controlling the actuation system 5 such that a specific ventilation maneuver, for example for recruiting the lung 19, is performed. As evident from the curve of the pressure p of the respiratory air when ventilating the patient shown in FIG. 3, the ventilation maneuver may be performed in such a way that initially an opening of the (previously closed) airways 17 and subsequently an opening of the (previously at least partially collapsed) lung 19 are implemented in an inhalation phase I of a single breath. Thereupon, there is a renewed closure, i.e. an at least partial collapse of the lung 19, in an exhalation phase E of the same breath. For example, the ventilation maneuver may have a duration of one minute or less.

The measurement data 11 are received in a step S2. The measurement data 11 may have been created when the ventilation maneuver was performed. In this example, the measurement data 11 disclose a pressure-dependent curve 27 of the carbon dioxide partial pressure pCO₂, referred to as pressure-dependent curve 27 for short below, said curve depending on the pressure p of the respiratory air breathed by the patient when the ventilation maneuver is performed (see FIG. 4). For example, the measurement data 11 may comprise (measured and/or estimated) values for at least one of the following measured quantities and/or may be based on (measured and/or estimated) values for at least one of the following quantities: the carbon dioxide partial pressure pCO₂, the oxygen partial pressure, the pressure p of the respiratory air, a volume V of the respiratory air, a volumetric flow rate Q of the respiratory air.

In a step S3, an airways opening pressure p_{o_aw} is determined for example by evaluating the pressure-dependent curve 27 of the inhalation phase I (indicated by a solid line in FIG. 4), in particular in the first half or the first third of the inhalation phase I. The airways opening pressure p_{o_aw} corresponds to a pressure p of the respiratory air in which there is a (complete) opening of the airways 17 (which for example closed during the previous exhalation).

In addition to that or in an alternative, a lung closing pressure p_{c_lu} that corresponds to a pressure p of the respiratory air at which the lung 19 (which for example opened during the previous inhalation) starts to collapse again may be determined by evaluating the pressure-dependent curve 27 of the exhalation phase E (indicated by a dashed line in FIG. 4), in particular in the last third of the exhalation phase E.

As shown in FIG. 3, it is also possible to determine a lung opening pressure p_{o_lu} by evaluating a time-dependent curve 28 of the volumetric flow rate Q of the respiratory air in the inhalation phase I, for example using the measurement data 11. The lung opening pressure p_{o_lu} may correspond to a pressure p of the respiratory air at which the volumetric flow rate Q of the respiratory air reaches its maximum in the inhalation phase I as a consequence of the (complete) opening of the lung 19 (which for example at least partially collapsed during the previous exhalation).

Expediently, the control signal 25 may be created such that an inhalation pressure p_{I} which corresponds to a pressure p of the respiratory air in the inhalation phase I is increased in a plurality of successive time steps. In this case, the inhalation pressure p_{I} in each time step may be higher than in the respective previous time step such that this results in a continuously increasing curve of the inhalation pressure p_{I}. In addition, the actuation system 5 may be controlled by means of the control signal 25 according to one of the predetermined inhalation pressure curves such that at least the majority of the inhalation pressure p_{I} increases linearly. Depending on the state of the respective patient, an at least partially nonlinear curve of the inhalation pressure p_{I} is also possible.

Analogous to the inhalation pressure p_{I}, the pressure p of the respiratory air in the exhalation phase E may be controlled in such a way by the control signal 25 that this results in a curve of the exhalation pressure pr that decreases continuously and/or at least largely linearly.

In order to increase the accuracy when evaluating the pressure-dependent curve 27 in the exhalation phase E, the respiratory air flow 7 may also be provided under flow control in the exhalation phase E. To this end, the control signal 25 may be created such that the volumetric flow rate Q of the respiratory air in the exhalation phase E follows a predetermined, e.g., constant volumetric flow rate. Suitable target values for the volumetric flow rate Q of the respiratory air are for example from about 0.02 l/s to about 0.20 l/s, preferably from about 0.05 l/s to about 0.15 l/s, and particularly preferably about 0.10 l/s. In principle, the volumetric flow rate Q in the exhalation phase E should not be too large so that a sufficiently large time window is available for evaluating the pressure-dependent curve 27.

As indicated by way of two gradient tangents placed against the pressure-dependent curve 27 in the example of the lung closing pressure p_{c_lu} in FIG. 4, an average gradient of the pressure-dependent curve 27 may be ascertained in a plurality of successive time steps, and a characteristic knee in the pressure-dependent curve 27 may be identified on the basis of the characteristic reduction in this gradient as the ventilation duration t increases. Depending on the time section of the inhalation phase I or exhalation phase E in which it occurs, this knee may indicate the airways opening pressure p_{o_aw} or the lung closing pressure p_{c_lu}.

If the airways 17 are closed and the inhalation pressure p_{I} reaches a critical threshold, then the airways 17 open suddenly such that fresh, i.e. oxygen-rich, respiratory air may flow into the lung 19. This leads to a dilution of the consumed, i.e. carbon-dioxide-rich, respiratory air that remained in the respiratory system 15 during the preceding exhalation, and this is expressed in a correspondingly significant drop in the carbon dioxide partial pressure pCO₂ in the respiratory air flow 7. The pressure p of the respiratory air associated with this knee in the pressure-dependent curve 27 may be considered to be the airways opening pressure p_{o_aw}.

As shown in FIG. 5, the measurement data 11 may additionally indicate a volume-dependent curve 29 of the carbon dioxide partial pressure pCO₂, referred to as a volume-dependent curve 29 for short below, as a function of the volume V of the respiratory air. The volume-dependent curve 29 may be used for an additional plausibility check when evaluating the pressure-dependent curve 27. To this end, a lung closing volume V_{c_lu} that corresponds to a volume V of the respiratory air in the at least partially collapsed lung 19 at or near the end of the exhalation phase E may be determined, for example on the basis of the volume-dependent curve 29. The lung closing volume V_{c_lu} and the lung closing pressure p_{c_lu} may correlate with each other in time, and so the two quantities are suitable for a mutual plausibility check.

Moreover, at least one target value for the pressure p of the respiratory air may be determined in a step S4 when the airways opening pressure p_{o_aw} and/or the lung opening pressure p_{o_lu} and/or the lung closing pressure p_{c_lu} are taken into account. The at least one target value may for example comprise an upper target value and a lower target value, between which the pressure p of the respiratory air should be varied.

Finally, the target value or the target values may be used in a step S5 for the further control of the actuation system 5 in a normal ventilation operation, i.e. following the ventilation maneuver.

Finally, it will be noted that terms such as “have”, “comprise”, “include”, “with” etc. do not exclude other elements or steps, and indefinite articles such as “a” or “an” do not exclude a plurality.

Furthermore, it will be noted that features or steps described with reference to one of the embodiments above can also be used in combination with features or steps described with reference to other embodiments from among the embodiments above.

### CITATIONS

1. Chen L, Del Sorbo L, Grieco D L, Shklar O, Junhasavasdikul D, Telias I, Fan E, Brochard L. Airway closure in acute respiratory distress syndrome: an underestimated and misinterpreted phenomenon. American Journal of Respiratory and Critical Care Medicine. 2018 Jan. 1; 197(1): 132-6.
2. Chen L, Del Sorbo L, Grieco D L, Junhasavasdikul D, Rittayamai N, Soliman I, Sklar M C, Rauseo M, Ferguson N D, Fan E, Richard J C. Potential for lung recruitment estimated by the recruitment-to-inflation ratio in acute respiratory distress syndrome. A clinical trial. American Journal of Respiratory and Critical Care Medicine. 2020 Jan. 15; 201(2): 178-87.
3. Beloncle F M, Richard J C, Merdji H, Desprez C, Pavlovsky B, Yvin E, Piquilloud L, Olivier P Y, Chean D, Studer A, Courtais A. Advanced respiratory mechanics assessment in mechanically ventilated obese and non-obese patients with or without acute respiratory distress syndrome. Critical Care. 2023 Sep. 4; 27(1): 343.
4. Chatburn R, Cutro R, Garnero A, A Babic S, Hatipoğlu U. Bedside assessment of lung recruitability: making sense of the recruitment-to-inflation ratio. Respiratory Care. 2023 October 68(10): 1465-1472.
5. Junhasavasdikul D, Kasemchaiyanun A, Tassaneyasin T et al. Expiratory flow limitation during mechanical ventilation: real-time detection and physiological subtypes. Critical Care 28, 171 (2024). https://doi.org/10.1186/s13054-024-04953-9

List of reference numerals

| | |
|---|---|
| 1 | Ventilator |
| 3 | Respiratory air connector |
| 5 | Actuation system |
| 7 | Respiratory air flow |
| 9 | Sensor system |
| 11 | Measurement data |
| 13 | Control unit |
| 15 | Respiratory system |
| 17 | Airways |
| 19 | Lung |
| 21 | Processor |
| 23 | Memory |
| 25 | Control signal |
| 27 | Pressure-dependent curve |
| 28 | Time-dependent curve |
| 29 | Volume-dependent curve |
| p | Pressure |
| p_{c}_{_{—}}ₗᵤ | Lung closing pressure |
| pₒ_{_{—}}_{aw} | Airways opening pressure |
| pₒ_{_{—}}ₗᵤ | Lung opening pressure |
| p_{E} | Exhalation pressure |
| p_{I} | Inhalation pressure |
| pCO₂ | Carbon dioxide partial pressure |
| t | Time, ventilation duration |
| E | Exhalation phase |
| I | Inhalation phase |
| M | Method |
| Q | Volumetric flow rate |
| S1 | Creating a control signal |
| S2 | Receiving measurement data |
| S3 | Determining an opening and/or closing pressure |
| S4 | Determining one or more target values |
| S5 | Using one or more target values |
| V | Volume |
| V_{c}_{_{—}}ₗᵤ | Lung closing volume |

What is claimed is:

1. A control unit for a ventilator, the ventilator comprising:
a respiratory air connector, to which a respiratory system of a patient is connected such that a ventilation of the patient with respiratory air is rendered possible;
an actuation system for providing a respiratory air flow at the respiratory air connector;
a sensor system for creating measurement data in relation to the ventilation;
wherein the control unit is configured to carry out the following method (M):
creating (S1) a control signal for controlling the actuation system such that a ventilation maneuver is performed, within the scope of which at least one section of the respiratory system is opened in an inhalation phase (I) and/or is closed in an exhalation phase (E);
receiving (S2) the measurement data, wherein the measurement data indicate a pressure-dependent curve of an amount of at least one respiratory gas inhaled by the patient in the inhalation phase (I) and/or exhaled in the exhalation phase (E), as a function of a pressure (p, p_{E}, p_{I}) of the respiratory air;
determining (S3) an opening pressure (p_{o_aw}, p_{o_lu}), which corresponds to a pressure (p, p_{I}) of respiratory air at which at least one section of the respiratory system is opened, by evaluating a section of the pressure-dependent curve that relates to the inhalation phase (I), and/or determining (S3) a closing pressure (p_{c_lu}), which corresponds to a pressure (p, p_{E}) of the respiratory air at which at least one section of the respiratory system is closed, by evaluating a section of the pressure-dependent curve that relates to the exhalation phase (E).

2. The control unit of claim 1,
wherein the opening pressure (p_{o_aw}, p_{o_lu}) comprises: an airways opening pressure (p_{o_aw}) that corresponds to a pressure (p, p_{I}) of the respiratory air at which the airways of the patient are at least partially opened, and/or a lung opening pressure (p_{o_lu}) that corresponds to a pressure (p, p_{I}) of the respiratory air at which a lung of the patient is at least partially opened; and/or
wherein the closing pressure (p_{c_lu}) comprises a lung closing pressure (p_{c_lu}) that corresponds to a pressure (p, p_{E}) of the respiratory air at which the lung of the patient is at least partially closed.

3. The control unit of claim 1,
wherein the control signal is created such that an inhalation pressure (p_{I}), which corresponds to a pressure (p) of the respiratory air in the inhalation phase (I), follows a predetermined inhalation pressure curve that increases linearly at least in part and/or is increased in a plurality of successive time steps, wherein the inhalation pressure (p_{I}) in each time step is higher than in the respective preceding time step.

4. The control unit of claim 1,
wherein the control signal is created such that a volumetric flow rate (Q) of the respiratory air in the exhalation phase (E) follows a predetermined volumetric flow rate; and/or
wherein a volumetric flow rate (Q) of the respiratory air in the exhalation phase (E) is from 0.02 l/s to 0.20 l/s.

5. The control unit of claim 1,
wherein the control signal is created such that an exhalation pressure (p_{E}), which corresponds to a pressure (p) of the respiratory air in the exhalation phase (E), follows a predetermined exhalation pressure curve that decreases linearly at least in part and/or is decreased in a plurality of successive time steps, wherein the exhalation pressure (p_{E}) in each time step is lower than in the respective preceding time step.

6. The control unit of claim 1,
wherein the determination (S3) of the opening pressure (p_{o_aw}, p_{o_lu}) and/or of the closing pressure (p_{c_lu}) comprises:
identifying a characteristic knee in the pressure-dependent curve;
determining a pressure (p, p_{E}, p_{I}) of the respiratory air associated with the characteristic knee, in order to determine the opening pressure (p_{o_aw}, p_{o_lu}) and/or the closing pressure (p_{c_lu}).

7. The control unit of claim 6,
wherein the characteristic knee is identified on the basis of a characteristic absolute-value reduction in an average gradient of the pressure-dependent curve with increasing ventilation duration (t).

8. The control unit of claim 1,
wherein the inhalation phase (I) and the exhalation phase (E) are successive phases in a single breath; and/or
wherein a section of the pressure-dependent curve is evaluated in the first half or in the first third of the inhalation phase (I) in order to determine (S3) the opening pressure (p_{o_aw}, p_{o_lu}), and/or a section of the pressure-dependent curve is evaluated in the last third of the exhalation phase (E) in order to determine (S3) the closing pressure (p_{c_lu}).

9. The control unit (13) of claim 1,
wherein the measurement data further indicate a volume-dependent curve of an amount of the at least one respiratory gas inhaled by the patient in the inhalation phase (I) and/or exhaled in the exhalation phase (E), as a function of a volume (V) of the respiratory air, wherein the opening pressure (p_{o_aw}, p_{o_lu}) is further determined by evaluating a section of the volume-dependent curve that relates to the inhalation phase (I), and/or the closing pressure (p_{c_lu}) is further determined by evaluating a section of the volume-dependent curve that relates to the exhalation phase (E).

10. The control unit of claim 1,
wherein the measurement data further indicate a time-dependent curve of a volumetric flow rate (Q) of the respiratory air inhaled by the patient in the inhalation phase (I) and/or exhaled in the exhalation phase (E), as a function of a ventilation duration (t), wherein the opening pressure (p_{o_aw}, p_{o_lu}) is further determined by evaluating a section of the time-dependent curve that relates to the inhalation phase (I), and/or the closing pressure (p_{c_lu}) is further determined by evaluating a section of the time-dependent curve that relates to the exhalation phase (E).

11. The control unit of claim 1,
wherein the method (M) further comprises:
determining (S4) at least one target value for the pressure (p, p_{E}, p_{I}) of the respiratory air taking into account the opening pressure (p_{o_aw}, p_{o_lu}) and/or the closing pressure (p_{c_lu});
using (S5) the at least one target value for controlling the actuation system.

12. The control unit of claim 1,
wherein the measurement data comprise values for at least one of the following quantities and/or are based on values for at least one of the following quantities: amount of the at least one respiratory gas inhaled by the patient in the inhalation phase (I) and/or exhaled in the exhalation phase (E), pressure (p, p_{E}, p_{I}) of the respiratory air, volume (V) of the respiratory air, volumetric flow rate (Q) of the respiratory air.

13. A ventilator, wherein the ventilator comprises:
a respiratory air connector for connecting a respiratory system of a patient such that a ventilation of the patient with respiratory air is rendered possible;
an actuation system for providing a respiratory air flow at the respiratory air connector;
a sensor system for creating measurement data in relation to the ventilation;
the control unit of claim 1.

14. A computer program for operating the ventilator of claim 13, wherein the computer program comprises commands that prompt the control unit to carry out the following method (M) when the computer program is executed by the control unit:
creating (S1) a control signal for controlling the actuation system such that a ventilation maneuver is performed, within the scope of which at least one section of the respiratory system is opened in an inhalation phase and/or is closed in an exhalation phase (E);
receiving (S2) the measurement data, wherein the measurement data indicate a pressure-dependent curve of an amount of at least one respiratory gas inhaled by the patient in the inhalation phase (I) and/or exhaled in the exhalation phase (E), as a function of a pressure (p, p_{E}, p_{I}) of the respiratory air;
determining (S3) an opening pressure (p_{o_aw}, p_{o_lu}), which corresponds to a pressure (p, p_{I}) of the respiratory air at which at least one section of the respiratory system is opened, by evaluating a section of the pressure-dependent curve that relates to the inhalation phase (I), and/or determining (S3) a closing pressure (p_{c_lu}), which corresponds to a pressure (p, p_{E}) of the respiratory air at which at least one section of the respiratory system is closed, by evaluating a section of the pressure-dependent curve that relates to the exhalation phase (E).

15. A computer-readable medium on which the computer program of claim 14 is stored.
